(19) United States
(12) Patent Application Publication
(10) Pub. No.: US 2025/0108028 A1
(43) Pub. Date: Apr. 3, 2025

(54) POLYUNSATURATED FATTY ACID MONOGLYCERIDES, COMPOSITIONS, METHODS AND USES THEREOF

(71) Applicant: SCF PHARMA INC., SAINTE-LUCE (CA)

(72) Inventor: Samuel C. Fortin, Sainte-Luce (CA)

(73) Assignee: SCF PHARMA INC., SAINTE-LUCE, QC (CA)

(21) Appl. No.: 18/725,239

(22) PCT Filed: Dec. 7, 2022
(86) PCT No.: PCT/CA2022/051785

§ 371 Date: Jun. 28, 2024

Related U.S. Application Data
(60) Provisional application No. 63/294,375, filed on Dec. 28, 2021.

(51) Int. Cl. A61K 31/232; A61K 31/353; A61K 45/06; A61P 39/00

(52) U.S. Cl. CPC A61K 31/232; A61K 31/353; A61K 45/06; A61P 39/00

(57) ABSTRACT

There are provided various compounds and compositions comprising polyunsaturated fatty acid monoglycerides and derivatives thereof. These compounds and compositions can be useful for use in cellular senescence prevention in a subject in need thereof or for slowing/delaying cellular senescence in a subject in need thereof. These compounds and compositions comprise at least one compound chosen from

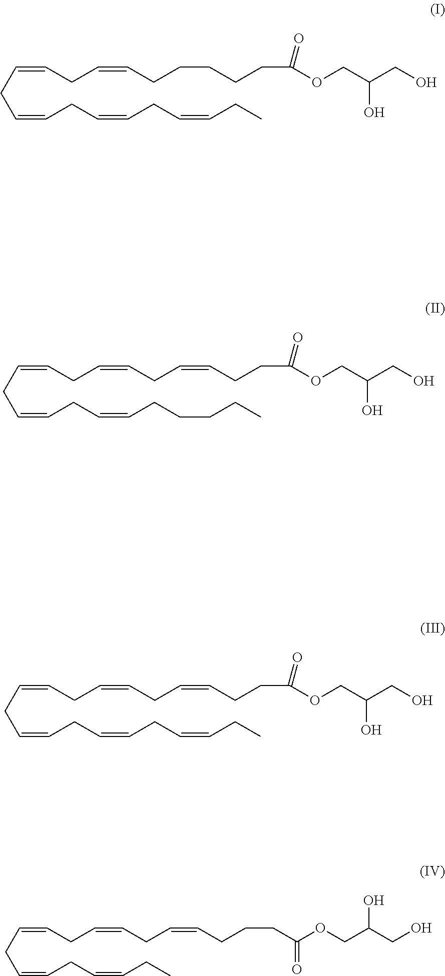 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]

### CROSS-REFERENCE TO RELATED APPLICATIONS

The present application claims priority to U.S. application No. 63/294,375 filed on Dec. 28, 2021. This document is hereby incorporated by reference in its entirety.

### FIELD OF THE DISCLOSURE

The present document relates to the field of cellular senescence. More particularly, it relates to polyunsaturated fatty acid monoglyceride compounds and compositions thereof. It also provided a use as cellular senescence prevention agent and/or a method for slowing/delaying cellular senescence.

### BACKGROUND OF THE DISCLOSURE

For thousand years, the human being's quest to find the fountain of youth was in vain. But things have changed with the cellular senescence theory. First described in the early 1960s, the cellular senescence is a phenomenon characterized by the cessation of cell division. The senescent cells, commonly known as zombie cells, are associated with the onset and development of multiple chronic diseases. In 2021, Herbig U. and. al. (Aging Cell, 20: e13344) demonstrate that peripheral blood mononuclear cells (PBMCs) isolated from healthy humans increasingly display cells with high senescence-associated β-galactosidase (SA-βGal) activity with advancing donor age. In mice, the selective elimination of senescent cells, extends median lifespan (Baker D J et al., Nature 479, 232-236 (2011)). It's now clear that removing or preventing senescent cell formation will lead to rejuvenation.

There are two strategies to avoid senescent cells, prevent them to form or kill them all with a senolytic like fisetin, quercetin or dasatinib. In recent years, most of the research on senescence is focused to find new senolytic agents to the detriment of prevention of senescent cells formation. Knowing that cellular senescence can be triggered by a DNA damage response due to the elevated reactive oxygen species (ROS) concentration in cells, a good strategy of prevention could be the intracellular ROS management.

### SUMMARY OF THE DISCLOSURE

According to one aspect there is provided at least one compound chosen from compound of formula (I), compound of formula (II), compound of formula (III) and compound of formula (IV):

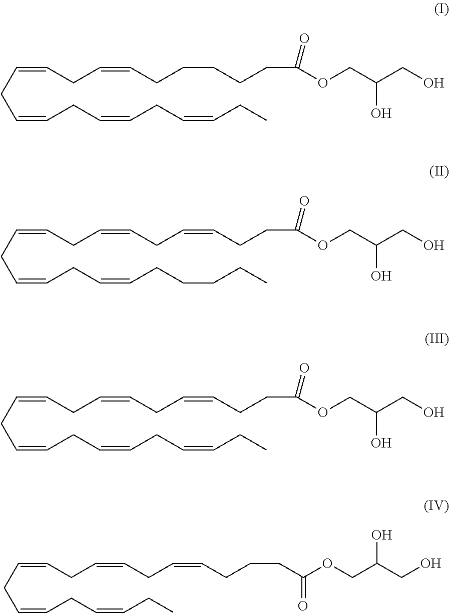 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]

for use in cellular senescence prevention in a subject in need thereof.

According to one aspect there is provided at least one compound chosen from compound of formula (I), compound of formula (II), compound of formula (III) and compound of formula (IV):

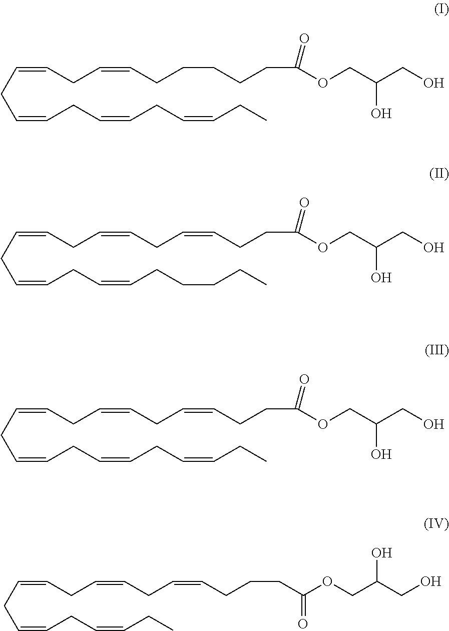 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]

for use as cellular senescence prevention agent in a subject in need thereof.

According to another aspect there is provided the use of at least one compound chosen from compound of formula (I), compound of formula (II), compound of formula (III) and compound of formula (IV) for use in slowing/delaying cellular senescence in a subject in need thereof.

According to another aspect there is provided the use of at least one compound chosen from compound of formula (I), compound of formula (II), compound of formula (III) and compound of formula (IV) in the manufacture of a medicament for cellular senescence prevention in a subject in need thereof.

According to another aspect there is provided the use of at least one compound chosen from compound of formula (I), compound of formula (II), compound of formula (III) and compound of formula (IV) in the manufacture of a medicament for slowing/delaying cellular senescence in a subject in need thereof.

According to another aspect there is provided a method for use in cellular senescence prevention in a subject in need thereof comprising administering an effective amount of at least one compound chosen from compound of formula (I), compound of formula (II), compound of formula (III) and compound of formula (IV).

According to another aspect there is provided a method for slowing/delaying cellular senescence of a subject in need thereof comprising administering to the subject an effective amount of at least one compound chosen from compound of formula (I), compound of formula (II), compound of formula (III) and compound of formula (IV).

According to another aspect, there is provided a composition comprising:
(i) at least one compound chosen from compound of formula (I), compound of formula (II), compound of formula (III) and compound of formula (IV);
(ii) at least one lipid.

According to another aspect, there is provided a composition comprising (i) at least one compound chosen from compound of formula (I), compound of formula (II), compound of formula (III) and compound of formula (IV) and (ii) at least one lipid for use in cellular senescence prevention in a subject in need thereof.

According to another aspect, there is provided a composition comprising (i) at least one compound chosen from compound of formula (I), compound of formula (II), compound of formula (III) and compound of formula (IV) and (ii) at least one lipid for slowing/delaying cellular senescence of a subject in need thereof.

According to another aspect, there is provided a composition comprising:
(i) at least one compound chosen from compound of formula (I), compound of formula (II), compound of formula (III) and compound of formula (IV);
(ii) at least one polyphenol.

According to another aspect, there is provided a composition comprising (i) at least one compound chosen from compound of formula (I), compound of formula (II), compound of formula (III) and compound of formula (IV) and (ii) at least one polyphenol for cellular senescence prevention in a subject in need thereof.

According to another aspect, there is provided a composition comprising (i) at least one compound chosen from compound of formula (I), compound of formula (II), compound of formula (III) and compound of formula (IV) and (ii) at least one polyphenol for slowing/delaying cellular senescence of a subject in need thereof

### BRIEF DESCRIPTION OF THE FIGURES

Further features and advantages will become more readily apparent from the following description of specific embodiments as illustrated by way of examples in the appended figures wherein.

FIG. 1 represents the effect of composition 1 comprising compound IV (Eicosapentaenoic acid monoglyceride, MAG-EPA) on senescence prevention ex-vivo assay using peripheral blood mononuclear cells (PBMCs) isolated from healthy human.

FIG. 2 represents the effect of composition 2 comprising compound IV (Eicosapentaenoic acid monoglyceride, MAG-EPA) and the polyphenol epigallocatechin gallate (EGCG) on senescence prevention ex-vivo assay using peripheral blood mononuclear cells (PBMCs) isolated from healthy human.

FIG. 3 represents the effect of separate administration compound IV (Eicosapentaenoic acid monoglyceride, MAG-EPA) and a composition of polyphenols (Fisetin, Quercetin and Epigallocatechin gallate) on a clinical case study of slowing/delaying cellular senescence.

### DETAILED DESCRIPTION OF THE DISCLOSURE

Further features and advantages of the previously-mentioned compounds will become more readily apparent from the following description of non-limiting examples.

The term “polyphenol” as used herein refers to as any organic molecule containing a plurality of phenol units, such as flavonoids anthocyanidins, flavan-3-ols (ex. Epigallocatechin gallate), Fisetin, flavonols (ex. Quercetin), flavanones, flavones, and isoflavones, tannic acid (quercitannic acid and gallotannic acid), the Blend of Fisetin/Quercetin (for example 1:5), microalgae polyphenol, macroalgae polyphenol, plants polyphenol, protists polyphenol and pharmaceutically acceptable salts thereof.

The term “microalgae polyphenol” as used herein refers to as any organic extract from a microalgae containing molecules with a plurality of phenol units, such as flavonoids anthocyanidins, flavan-3-ols (ex. Epigallocatechin gallate), Fisetin, flavonols (ex. Quercetin), flavanones, flavones, and isoflavones, tannic acid (quercitannic acid and gallotannic acid), and pharmaceutically acceptable salts thereof.

The term “macroalgae polyphenol” as used herein refers to as any organic extract from a macroalgae containing molecules with a plurality of phenol units, such as flavonoids anthocyanidins, flavan-3-ols (ex. Epigallocatechin gallate), Fisetin, flavonols (ex. Quercetin), flavanones, flavones, and isoflavones, tannic acid (quercitannic acid and gallotannic acid), and pharmaceutically acceptable salts thereof.

The term “plants polyphenol” as used herein refers to as any organic extract from a plant containing molecules with a plurality of phenol units, such as flavonoids anthocyanidins, flavan-3-ols (ex. Epigallocatechin gallate), Fisetin, flavonols (ex. Quercetin), flavanones, flavones, and isoflavones, tannic acid (quercitannic acid and gallotannic acid), and pharmaceutically acceptable salts thereof.

The term “protists polyphenol” as used herein refers to as any organic extract from a protist containing molecules with a plurality of phenol units, such as flavonoids anthocyanidins, flavan-3-ols (ex. Epigallocatechin gallate), Fisetin, flavonols (ex. Quercetin), flavanones, flavones, and isoflavones, tannic acid (quercitannic acid and gallotannic acid), and pharmaceutically acceptable salts thereof.

The term “lipid” as used herein refers to as any fat-soluble (lipophilic), molecules, such as fats, fat-like substances, oils (such as animal oil, marine oil, vegetable oil, fish oil concentrate, re-esterified fish oil or re-esterified concentrated fish oil), waxes, sterols (such as cholesterol, ergosterol, sitosterol, stigmasterol, fat-soluble vitamins (such as vitamins A, D, E and K), fatty acids, oxidized fatty acid (such as lipoxin, specialized pro-resolving mediators or epoxydes), fatty acids esters thereof, and various derivatives thereof such as monoglycerides, diglycerides, triglycerides, phospholipids, glycolipids, and cerebrosides and pharmaceutically acceptable salts thereof.

The expression “effective amount” of a compound of the present disclosure is a quantity sufficient to, when administered to the subject, including a mammal, for example a human, effect beneficial or desired results, including clinical results, and, as such, an “effective amount” depends upon the context in which it is being applied. The amount of a given compound of the present disclosure that will correspond to such an amount will vary depending upon various factors, such as the given drug or compound, the pharmaceutical formulation, the route of administration, the identity of the subject or host being treated, and the like, but can nevertheless be routinely determined by one skilled in the art.

The expression “separate administration” or “be administered separately” of the present disclosure refers to a therapy combining the administration of compound of formula (I), (II), (III) or (IV) for 1 day and then another compound (for example polyphenol) for the next day. The therapy can also be the administration of compound of formula (I), (II), (III) or (IV) for 1 week and another compound (for example polyphenol) for the next week. It can also be the administration of compound of formula (I), (II), (III) or (IV) for 1 month and another compound (for example polyphenol) for the next month. Several other combinations can be used: 1 month/1 day; 1 month/2 days; 1 month/1 week; 2 months/1 day: 2 months/2 days; 2 months/1 week; 2 months/2 weeks; 3 months/1 day; 3 months/2 days; 3 months/1 week; 3 months/2 weeks; 3 months/3 weeks; 4 months/2 days; 4 months/1 week: 4 months/2 weeks; 4 months/3 weeks; 4 months/1 month; 6 months/1 month; 7 months/1 month; 8 months/1 month; 9 months/1 month; 10 months/1 month; 11 months/1 month; 1 year/1 month; 1 year/2 months or 1 year/3 months.

For example, the subject in need thereof can be a bee, human, cat, dog, etc.

For example, the at least one compound is the compound of formula (I).

For example, the at least one compound is the compound of formula (II).

For example, the at least one compound is the compound of formula (III).

For example, the at least one compound is the compound of formula (IV).

For example, the at least one compound is the compound of formula (I), the compound of formula (III) and the compound of formula (IV).

For example, the at least one compound is the compound of formula (I) and the compound of formula (IV).

For example, the at least one compound is the compound of formula (I) and the compound of formula (III).

For example, the at least one compound is the compound of formula (III) and the compound of formula (IV).

For example, the at least one compound can be for use in combination with at least one lipid.

For example, the at least one compound can be for use in combination with at least one polyphenol

For example, the at least one lipid and the at least one compound can be for simultaneous administration.

For example, the at least one polyphenol and the at least one compound can be for simultaneous administration.

For example, the at least one lipid and the at least one compound can be for separate administration.

For example, the at least one polyphenol and the at least one compound can be for separate administration.

For example, the at least one compound chosen from compound of formula (I), compound of formula (II), compound of formula (III) and compound of formula (IV) can be administered in combination with at least one lipid.

For example, the at least one compound chosen from compound of formula (I), compound of formula (II), compound of formula (III) and compound of formula (IV) can be administered in combination with at least one polyphenol.

For example, the at least one lipid and the at least one compound can be administered simultaneously.

For example, the at least one polyphenol and the at least one compound can be administered simultaneously.

For example, the at least one lipid and the at least one compound can be administered separately.

For example, the at least one polyphenol and the at least one compound can be administered separately.

For example, the at least one lipid can be chosen from animal oil, marine oil, fish oil, fish oil concentrate, re-esterified fish oil, re-esterified concentrated fish oil, vegetable oil, fatty acids, fatty acids ethyl esters, fatty acids esters, monoglycerides, diglycerides, triglycerides and phospholipids.

For example, the at least one lipid can be chosen from marine oil, fish oil, fish oil concentrate, re-esterified fish oil, re-esterified concentrated fish oil, fatty acids ethyl esters, fatty acids esters, monoglycerides, diglycerides, triglycerides and phospholipids.

For example, the at least one polyphenol can be chosen from flavonoids anthocyanidins, flavan-3-ols, epigallocatechin gallate, fisetin, flavonols, quercetin, flavanones, flavones, isoflavones, tannic acid, quercitannic acid, gallotannic acid and mixtures thereof. For example, the at least one polyphenol can be a mixture of Fisetin/Quercetin (1:5).

For example, the at least one polyphenol can be chosen from epigallocatechin gallate, fisetin, quercetin, and mixtures thereof.

For example, the at least one polyphenol can be chosen from microalgae polyphenol, macroalgae polyphenol, plants polyphenol and protists polyphenol.

For example, the slowing/delaying cellular senescence can comprise slowing/delaying cellular senescence of peripheral blood mononuclear cells.

For example, the slowing/delaying cellular senescence comprises decreasing % of senescent peripheral blood mononuclear cells.

For example, the slowing/delaying cellular senescence comprises decreasing at least about 25% of senescent peripheral blood mononuclear cells in a period of time of about 21 days, about 14 days or about 7 days.

For example, the slowing/delaying cellular senescence comprises decreasing at least about 25% of senescent peripheral blood mononuclear cells in a period of time of about 7 days to about 21 days.

For example, the slowing/delaying cellular senescence comprises decreasing at least about 33% of senescent peripheral blood mononuclear cells in a period of time of about 21 days, about 14 days or about 7 days.

For example, the slowing/delaying cellular senescence comprises decreasing at least about 33% of senescent peripheral blood mononuclear cells in a period of time of about 7 days to about 21 days.

For example, the compound chosen from compound of formula (I), compound of formula (II), compound of formula (III) and compound of formula (IV) is administered to a patient in need thereof at a suitable amount.

For example, the compound chosen from compound of formula (I), compound of formula (II), compound of formula (III) and compound of formula (IV) is administered to a patient in need thereof in an amount of about 50 mg/day to about 5000 mg/day.

For example, the compound chosen from compound of formula (I), compound of formula (II), compound of formula (III) and compound of formula (IV) is administered to a patient in need thereof in an amount of about 50 mg/day to about 500 mg/day.

For example, the compound chosen from compound of formula (I), compound of formula (II), compound of formula (III) and compound of formula (IV) is administered to a patient in need thereof in an amount of about 1 mg/kg/day to about 100 mg/kg/day.

For example, the compound chosen from compound of formula (I), compound of formula (II), compound of formula (III) and compound of formula (IV) is administered to a patient in need thereof in an amount of about 1 mg/kg/day to about 50 mg/kg/day.

For example, the compound chosen from compound of formula (I), compound of formula (II), compound of formula (III) and compound of formula (IV) is administered to a patient in need thereof in an amount of about 2 mg/kg/day to about 20 mg/kg/day.

For example, the compound chosen from compound of formula (I), compound of formula (II), compound of formula (III) and compound of formula (IV) is administered to a patient in need thereof in an amount of about 3 mg/kg/day to about 9 mg/kg/day.

For example, the compound chosen from compound of formula (I), compound of formula (II), compound of formula (III) and compound of formula (IV) is administered to a patient in need thereof in an amount of about 4 mg/kg/day to about 6 mg/kg/day.

For example, the compound chosen from compound of formula (I), compound of formula (II), compound of formula (III) and compound of formula (IV) is administered for a period of time of at least one month, at least two months, at least three months, at least four months, at least five months or at least six months.

For example, the compound chosen from compound of formula (I), compound of formula (II), compound of formula (III) and compound of formula (IV) is administered for a period of time of about 1 month to about 12 months.

For example, the at least one polyphenol is administered to a patient in need thereof in a suitable amount.

For example, the at least one polyphenol is administered to a patient in need thereof in an amount of about 50 mg/day to about 5000 mg/day.

For example, the at least one polyphenol is administered to a patient in need thereof in an amount of about 50 mg/day to about 500 mg/day.

For example, the at least one polyphenol is administered to a patient in need thereof in an amount of about 1 mg/kg/day to about 100 mg/kg/day.

For example, the at least one polyphenol is administered to a patient in need thereof in an amount of about 1 mg/kg/day to about 50 mg/kg/day.

For example, the at least one polyphenol is administered to a patient in need thereof in an amount of about 2 mg/kg/day to about 20 mg/kg/day.

For example, the at least one polyphenol is administered to a patient in need thereof in an amount of about 2 mg/kg/day to about 20 mg/kg/day.

For example, the at least one polyphenol is administered to a patient in need thereof in an amount of about 3 mg/kg/day to about 9 mg/kg/day.

For example, the at least one polyphenol is administered to a patient in need thereof in an amount of about 4 mg/kg/day to about 6 mg/kg/day.

For example, the at least one polyphenol is administered for a period of time of at least 2 days, at least 5 days, at least 7 days, at least 10 days or at least 14 days.

For example, the at least one polyphenol is administered for a period of time of about 2 days to about 15 days.

For example, the at least one polyphenol is administered for a period of time of about 1 days to about 30 days, about 1 days to about 21 days, about 2 days to about 14 days or about 7 days to about 14 days.

For example, the compound chosen from compound of formula (I), compound of formula (II), compound of formula (III) and compound of formula (IV) is administered to a patient in need thereof for a period of time of about 1 to 12 months, then the administration is discontinued and subsequent to discontinuation, the at least one polyphenol is administered for a period of time of about 1 day to about 15 days.

For example, the compound chosen from compound of formula (I), compound of formula (II), compound of formula (III) and compound of formula (IV) is administered to a patient in need thereof for a period of time of about 1 to about 12 months or about 2 to about 6 months, then the administration is discontinued and subsequent to discontinuation, the at least one polyphenol is administered for a period of time of about 1 day to about 30 days, about 1 day to about 14 days or about 2 days to about 14 days.

For example, the compound chosen from compound of formula (I), compound of formula (II), compound of formula (III) and compound of formula (IV) is firstly administered to a patient in need thereof for a period of time of about 1 to 12 months, then administration of the compound chosen from compound of formula (I), compound of formula (II), compound of formula (III) and compound of formula (IV) is stopped, and then secondly, the at least one polyphenol is administered for a period of time of about 1 day to about 15 days.

For example, the compound chosen from compound of formula (I), compound of formula (II), compound of formula (III) and compound of formula (IV) is administered to a patient in need thereof for a period of time of about 1 to about 12 months or about 2 to about 6 months, then administration of the compound chosen from compound of formula (I), compound of formula (II), compound of formula (III) and compound of formula (IV) is stopped, and then secondly, the at least one polyphenol is administered for a period of time of about 1 day to about 30 days, about 1 day to about 14 days or about 2 days to about 14 days.

For example, the composition or formulation can comprise about 1 mg to about 3000 mg, about 5 mg to about 2500 mg, about 10 mg to about 2000 mg, about 75 mg to about 1100 mg, about 25 mg to about 2000 mg, about 25 mg to about 300 mg, about 50 mg to about 1500 mg, about 100 mg to about 1400 mg, about 50 mg to about 300 mg, about 10 mg to about 500 mg, about 10 mg to about 100 mg, about 10 mg to about 250 mg, about 1 mg to about 100 mg, about 1 mg to about 10 mg, about 5 mg to about 50 mg, about 5 mg to about 100 mg, about 0.1 mg to about 150 mg, about 10 mg to about 250 mg or about 100 mg to about 500 mg, of at least one compound chosen from compound of formula (I), compound of formula (II), compound of formula (III) and compound of formula (IV).

For example, the composition or formulation can comprise about 1 mg to about 3000 mg, about 5 mg to about 2500 mg, about 10 mg to about 2000 mg, about 75 mg to about 1100 mg, about 25 mg to about 2000 mg, about 25 mg to about 300 mg, about 50 mg to about 1500 mg, about 100 mg to about 1400 mg, about 50 mg to about 300 mg, about 10 mg to about 500 mg, about 10 mg to about 100 mg, about 10 mg to about 250 mg, about 1 mg to about 100 mg, about 1 mg to about 10 mg, about 5 mg to about 50 mg, about 5 mg to about 100 mg, about 0.1 mg to about 150 mg, about 10 mg to about 250 mg or about 100 mg to about 500 mg, of at least one polyphenol.

For example, the compound chosen from compound of formula (I), compound of formula (II), compound of formula (III) and compound of formula (IV) is administered to a patient in need thereof via 100 mg, 250 mg, 500 mg, 1000 mg or 2000 mg softgel capsules.

For example, the at least one polyphenol can be administered to a patient in need thereof via 100 mg, 250 mg, 500 mg, 1000 or 2000 mg tablets.

### Example 1

### Preparation of a Composition (Composition 1) Comprising Compound IV.

Composition 1 comprising compound IV, was prepared by reacting EPA concentrated fish oil (ethyl ester form) with glycerol with the lipase NOVOZYM 435™—in acetone. The final composition 1 comprises compound IV (40% w/w), unreacted ethyl ester (50% w/w) and small amount of diglycerides and triglycerides (10% w/w).

### Example 2

### Preparation of a Composition (Composition 2) Comprising Compound IV.

Composition 2 comprising compound IV (Eicosapentaenoic acid monoglyceride, MAG-EPA) and the polyphenol epigallocatechin gallate (EGCG), was prepared by mixing EGCG (10 mg) in composition 1 (10 mg) at room temperature.

### Example 3

### Composition 1 Prevents the Formation of Senescent Cells.

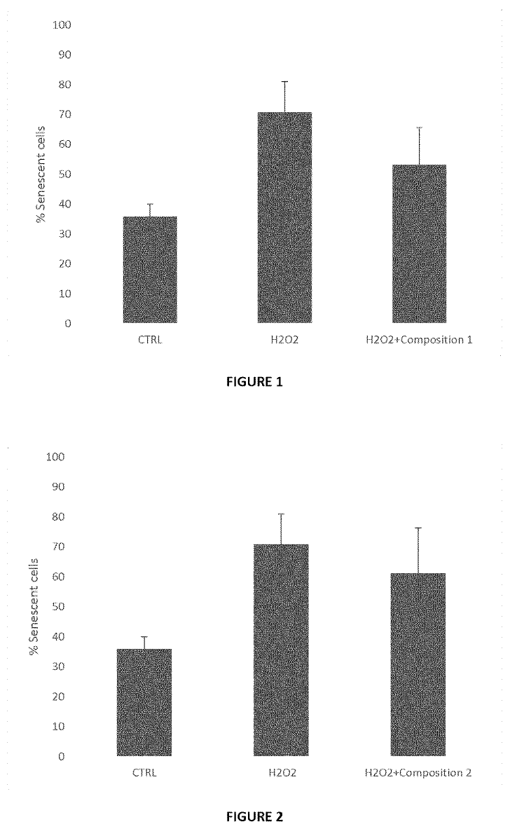

Blood samples were obtained from 1 healthy donor recruited at SCF Pharma after informed consent was obtained (IRB Tracking Number: 2020-2415-3291-1). PBMCs were isolated from whole blood by centrifugation on Lymphoprep™ in accordance with the manufacturer's recommendations. After isolation, PBMCs were washed 2 times in phosphate buffered saline (PBS). Cells were cultured using 5×10⁵ cells/well in 1 ml RPMI culture medium in the presence of 1% foetal bovine serum at 37° C. under 5% CO2. The cells were unstimulated (control) or stimulated for 2 h with 200 μM H₂O₂ to induce senescence. Composition 1 (5 μg/ml) was added 24 h before H₂O₂ treatment and the cells were incubated for 24 h in duplicate. Cell senescence quantification was performed using the β-galactosidase (SA-βGal) assay. FIG. 1 shows the senescence prevention action of composition 1 by reducing the percentage of senescent cells following induction by H₂O₂.

### Example 4

### Composition 2 Prevents the Formation of Senescent Cells.

Blood samples were obtained from 1 healthy donor recruited at SCF Pharma after informed consent was obtained (IRB Tracking Number: 2020-2415-3291-1). PBMCs were isolated from whole blood by centrifugation on Lymphoprep™ in accordance with the manufacturer's recommendations. After isolation, PBMCs were washed 2 times in phosphate buffered saline (PBS). Cells were cultured using 5×10⁵ cells/well in 1 ml RPMI culture medium in the presence of 1% foetal bovine serum at 37° C. under 5% CO2. The cells were unstimulated (control) or stimulated for 2 h with 200 μM H₂O₂ to induce senescence. Composition 2 (5 μg/ml) was added 24 h before H₂O₂ treatment and the cells were incubated for 24 h in duplicate. Cell senescence quantification was performed using the β-galactosidase (SA-βGal) assay. FIG. 2 shows the senescence prevention action of composition 2 by reducing the percentage of senescent cells following induction by H₂O₂.

### Example 5

Slowing/Delaying Human Peripheral Blood Mononuclear Cells (PBMCs) Senescence with a Separate Administration of Composition 1 and a Mixture of Polyphenols.

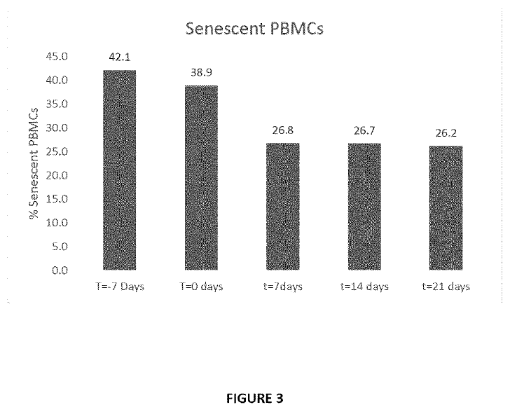

A pilot study was conducted on one human volunteer of about 80 kg who was on continuous omega-3 supplementation with 450 mg/day of compound IV for more than a year. The volunteer stopped the composition 1 supplementation at T=0 day and switch to a dosage of 1700 mg/day of a mixture of polyphenols (Fisetin 200 mg, Quercetin 1000 mg and Epigallocatechin gallate 500 mg) supplementation for 21 days. Blood samples were obtained at T=−7 days, 0 day, 7 days, 14 days and 21 days. PBMCs were isolated from whole blood by centrifugation on Lymphoprep™ in accordance with the manufacturer's recommendations. After isolation, purification and stabilization, the percentage of senescent PBMCs was assessed using the β-galactosidase (SA-βGal) assay. FIG. 3 shows the slowing/delaying effect, of a separate administration of composition 1 and a mixture of polyphenols, on senescence of human PBMCs over a four (4) week period. As it can be seen from FIG. 3, use of polyphenols allowed for a significant decrease of senescent PBMCs. In fact, after administration of polyphenols, it was possible to measure a decrease of about 33% (⅓) of the senescent PBMCs after only a week and this level of senescent cell was maintained for two more week during the polyphenols supplementation.

The separate administration of polyphenols and composition 1 is preferred to the simultaneously administration to decrease the amount of senescent cells. As shown in FIG. 1, composition 1 alone decrease the amount of senescent cells by 25% and with simultaneous administration with a polyphenol, the decrease was only 15% (FIG. 2). When a separate administration is used, the decrease of senescent cells reaches 33% (FIG. 3).

While the compounds, compositions, methods and uses thereof have been described in connection with specific embodiments thereof, it will be understood that they can be further modified and this application is intended to cover any variations, uses, or adaptations of the compounds, compositions, methods and uses thereof following, in general, the principles described in the present document and including such departures from the present disclosure as come within known or customary practice within the art to which the present document pertains and as may be applied to the features hereinbefore set forth, and as follows in the scope of the appended claims.

What is claimed is:

1. At least one compound chosen from compound of formula (I), compound of formula (II), compound of formula (III) and compound of formula (IV): 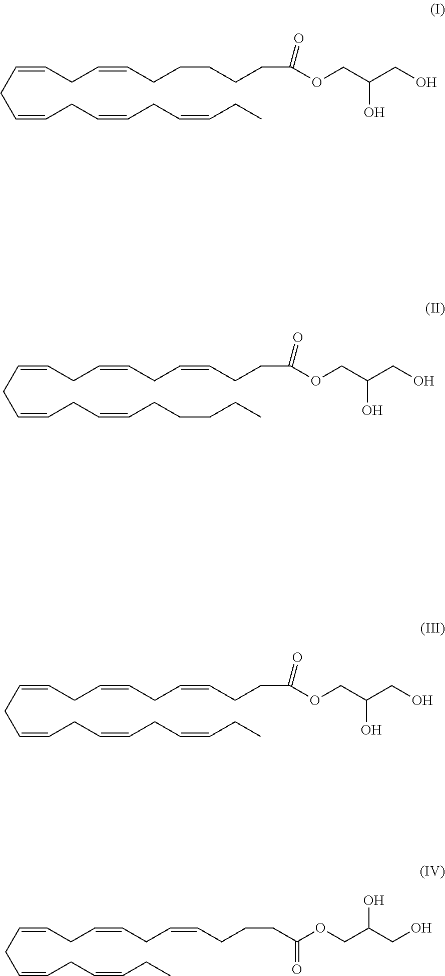 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]
for use in cellular senescence prevention in a subject in need thereof.

2. At least one compound chosen from compound of formula (I), compound of formula (II), compound of formula (III) and compound of formula (IV): 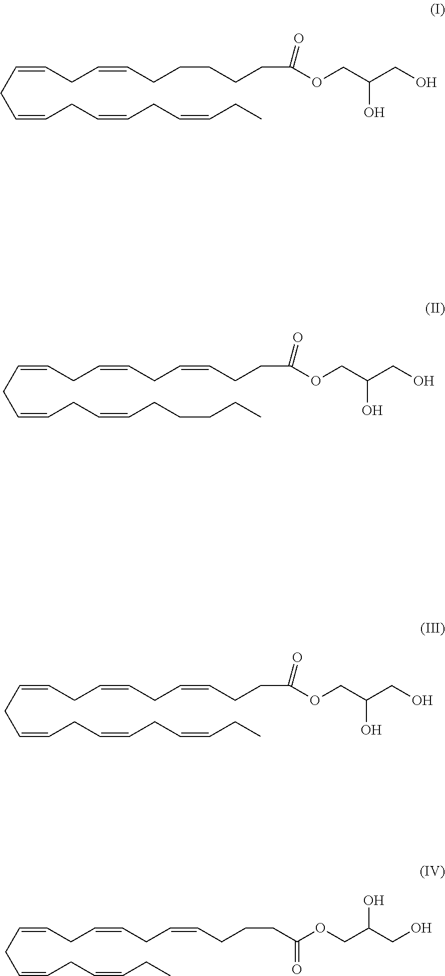 [SMILES: CC/C=C\C/C=C\C/C=C\C/C=C\C/C=C\CCCCCC(=O)OCC(O)CO ; CCCCC/C=C\C/C=C\C/C=C\C/C=C\C/C=C\CCC(=O)OCC(O)CO ; CC/C=C\C/C=C\C/C=C\C/C=C\C/C=C\C/C=C\CCC(=O)OCC(O)CO ; CC/C=C\C/C=C\C/C=C\C/C=C\C/C=C\CCCC(=O)OCC(O)CO]
for use as cellular senescence prevention agent in a subject in need thereof.

3. At least one compound chosen from compound of formula (I), compound of formula (II), compound of formula (III) and compound of formula (IV), for use in a method for slowing/delaying cellular senescence in a subject in need thereof.

4. At least one compound chosen from compound of formula (I), compound of formula (II), compound of formula (III) and compound of formula (IV), for use in slowing/delaying cellular senescence in a subject in need thereof.

5. The at least one compound of any one of claims 1 to 4, wherein said at least one compound is said compound of formula (I).

6. The at least one compound of any one of claims 1 to 4, wherein said at least one compound is said compound of formula (II).

7. The at least one compound of any one of claims 1 to 4, wherein said at least one compound is said compound of formula (III).

8. The at least one compound of any one of claims 1 to 4, wherein said at least one compound is said compound of formula (IV).

9. The at least one compound of any one of claims 1 to 4, wherein said at least one compound is said compound of formula (I), said compound of formula (III) and said compound of formula (IV).

10. The at least one compound of any one of claims 1 to 4, wherein said at least one compound is said compound of formula (I) and said compound of formula (IV).

11. The at least one compound of any one of claims 1 to 4, wherein said at least one compound is said compound of formula (I) and said compound of formula (III).

12. The at least one compound of any one of claims 1 to 4, wherein said at least one compound is said compound of formula (III) and said compound of formula (IV).

13. The at least one compound of any one of claims 1 to 12, for use in combination with at least one lipid.

14. The at least one compound of claim 13, wherein said at least one lipid and said at least one compound are for simultaneous administration.

15. The at least one compound of claim 13, wherein said at least one lipid and said at least one compound are for separate administration.

16. The at least one compound of any one of claims 1 to 12, for use in combination with at least one polyphenol.

17. The at least one compound of claim 16, wherein said at least one polyphenol and said at least one compound are for simultaneous administration.

18. The at least one compound of claim 16, wherein said at least one polyphenol and said at least one compound are for separate administration.

19. The at least one compound of claim 16, 17 or 18, wherein said at least one polyphenol is chosen from flavonoids anthocyanidins, flavan-3-ols, epigallocatechin gallate, fisetin, flavonols, quercetin, flavanones, flavones, isoflavones, tannic acid, quercitannic acid, gallotannic acid and mixtures thereof.

20. The at least one compound of claim 16, 17 or 18, wherein said at least one polyphenol is chosen from epigallocatechin gallate, fisetin, quercetin, and mixtures thereof.

21. The at least one compound of any one of claims 1 to 20, wherein the slowing/delaying cellular senescence comprises slowing/delaying cellular senescence of peripheral blood mononuclear cells.

22. The at least one compound of any one of claims 1 to 20, wherein the slowing/delaying cellular senescence comprises decreasing % of senescent peripheral blood mononuclear cells.

23. The at least one compound of any one of claims 1 to 20, wherein the slowing/delaying cellular senescence comprises decreasing at least about 25% of senescent peripheral blood mononuclear cells in a period of time of about 21 days.

24. The at least one compound of any one of claims 1 to 20, wherein the slowing/delaying cellular senescence comprises decreasing at least about 25% of senescent peripheral blood mononuclear cells in a period of time of about 14 days.

25. The at least one compound of any one of claims 1 to 20, wherein the slowing/delaying cellular senescence comprises decreasing at least about 25% of senescent peripheral blood mononuclear cells in a period of time of about 7 days.

26. The at least one compound of any one of claims 1 to 20, wherein the slowing/delaying cellular senescence comprises decreasing at least about 33% of senescent peripheral blood mononuclear cells in a period of time of about 21 days.

27. The at least one compound of any one of claims 1 to 20, wherein the slowing/delaying cellular senescence comprises decreasing at least about 33% of senescent peripheral blood mononuclear cells in a period of time of about 14 days.

28. The at least one compound of any one of claims 1 to 20, wherein the slowing/delaying cellular senescence comprises decreasing at least about 33% of senescent peripheral blood mononuclear cells in a period of time of about 7 days.

29. Use of at least one compound chosen from compound of formula (I), compound of formula (II), compound of formula (III) and compound of formula (IV), for cellular senescence prevention in a subject in need thereof.

30. Use of at least one compound chosen from compound of formula (I), compound of formula (II), compound of formula (III) and compound of formula (IV), as a cellular senescence prevention agent.

31. Use of at least one compound chosen from compound of formula (I), compound of formula (II), compound of formula (III) and compound of formula (IV), for slowing/delaying cellular senescence in a subject in need thereof.

32. Use of at least one compound chosen from compound of formula (I), compound of formula (II), compound of formula (III) and compound of formula (IV), in the manufacture of a medicament for cellular senescence prevention in a subject in need thereof.

33. Use of at least one compound chosen from compound of formula (I), compound of formula (II), compound of formula (III) and compound of formula (IV), in the manufacture of a medicament for slowing/delaying cellular senescence in a subject in need thereof.

34. The use of any one of claims 29 to 33, wherein said at least one compound is said compound of formula (I).

35. The use of any one of claims 29 to 33, wherein said at least one compound is said compound of formula (II).

36. The use of any one of claims 29 to 33, wherein said at least one compound is said compound of formula (III).

37. The use of any one of claims 29 to 33, wherein said at least one compound is said compound of formula (IV).

38. The use of any one of claims 29 to 33, wherein said at least one compound is said compound of formula (I), said compound of formula (III) and said compound of formula (IV).

39. The use of any one of claims 29 to 33, wherein said at least one compound is said compound of formula (I) and said compound of formula (IV).

40. The use of any one of claims 29 to 33, wherein said at least one compound is said compound of formula (I) and said compound of formula (III).

41. The use of any one of claims 29 to 33, wherein said at least one compound is said compound of formula (III) and said compound of formula (IV).

42. The use of any one of claims 29 to 33, for use in combination with at least one lipid.

43. The use of claim 34, wherein said at least one lipid and said at least one compound are for simultaneous administration.

44. The use of claim 42, wherein said at least one lipid and said at least one compound are for separate administration.

45. The use of any one of claims 29 to 41, for use in combination with at least one polyphenol.

46. The use of claim 45, wherein said at least one polyphenol and said at least one compound are for simultaneous administration.

47. The use of claim 45, wherein said at least one polyphenol and said at least one compound are for separate administration.

48. The use of claim 45, 46 or 47, wherein said at least one polyphenol is chosen from flavonoids anthocyanidins, flavan-3-ols, epigallocatechin gallate, fisetin, flavonols, quercetin, flavanones, flavones, isoflavones, tannic acid, quercitannic acid, gallotannic acid and mixtures thereof.

49. The use of claim 45, 46 or 47, wherein said at least one polyphenol is chosen from epigallocatechin gallate, fisetin, quercetin, and mixtures thereof.

50. The use of any one of claims 45 to 49, wherein said compound chosen from compound of formula (I), compound of formula (II), compound of formula (III) and compound of formula (IV) is administered to a patient in need thereof at a suitable amount.

51. The use of any one of claims 45 to 49, wherein said compound chosen from compound of formula (I), compound of formula (II), compound of formula (III) and compound of formula (IV) is administered to a patient in need thereof in an amount of about 50 mg/day to about 5000 mg/day.

52. The use of any one of claims 45 to 49, wherein said compound chosen from compound of formula (I), compound of formula (II), compound of formula (III) and compound of formula (IV) is administered to a patient in need thereof in an amount of about 50 mg/day to about 500 mg/day.

53. The use of any one of claims 45 to 49, wherein said compound chosen from compound of formula (I), compound of formula (II), compound of formula (III) and compound of formula (IV) is administered to a patient in need thereof in an amount of about 1 mg/kg/day to about 100 mg/kg/day.

54. The use of any one of claims 45 to 49, wherein said compound chosen from compound of formula (I), compound of formula (II), compound of formula (III) and compound of formula (IV) is administered to a patient in need thereof in an amount of about 1 mg/kg/day to about 50 mg/kg/day.

55. The use of any one of claims 45 to 49, wherein said compound chosen from compound of formula (I), compound of formula (II), compound of formula (III) and compound of formula (IV) is administered to a patient in need thereof in an amount of about 2 mg/kg/day to about 20 mg/kg/day.

56. The use of any one of claims 45 to 49, wherein said compound chosen from compound of formula (I), compound of formula (II), compound of formula (III) and compound of formula (IV) is administered for a period of time of at least 1 month.

57. The use of any one of claims 45 to 49, wherein said compound chosen from compound of formula (I), compound of formula (II), compound of formula (III) and compound of formula (IV) is administered for a period of time of at least 4 months.

58. The use of any one of claims 45 to 49, wherein said compound chosen from compound of formula (I), compound of formula (II), compound of formula (III) and compound of formula (IV) is administered for a period of time of about 1 month to about 12 months.

59. The use of any one of claims 45 to 58, wherein said at least one polyphenol is administered to a patient in need thereof at a suitable amount.

60. The use of any one of claims 45 to 58, wherein said at least one polyphenol is administered to a patient in need thereof in an amount of about 50 mg/day to about 5000 mg/day.

61. The use of any one of claims 45 to 58, wherein said compound chosen from compound of formula (I), compound of formula (II), compound of formula (III) and compound of formula (IV) is administered to a patient in need thereof in an amount of about 50 mg/day to about 500 mg/day.

62. The use of any one of claims 45 to 58, wherein said at least one polyphenol is administered to a patient in need thereof in an amount of about 1 mg/kg/day to about 100 mg/kg/day.

63. The use of any one of claims 45 to 58, wherein said at least one polyphenol is administered to a patient in need thereof in an amount of about 1 mg/kg/day to about 50 mg/kg/day.

64. The use of any one of claims 45 to 58, wherein said at least one polyphenol is administered to a patient in need thereof in an amount of about 2 mg/kg/day to about 20 mg/kg/day.

65. The use of any one of claims 45 to 64, wherein said at least one polyphenol is administered for a period of time of at least 2 days.

66. The use of any one of claims 45 to 64, wherein said at least one polyphenol is administered for a period of time of at least 10 days.

67. The use of any one of claims 45 to 64, wherein said at least one polyphenol is administered for a period of time of about 2 days to about 15 days.

68. The use of any one of claims 45 to 64, wherein said compound chosen from compound of formula (I), compound of formula (II), compound of formula (III) and compound of formula (IV) is administered to a patient in need thereof for a period of time of about 1 to 12 months, then said administration is discontinued and subsequent to discontinuation, said at least one polyphenol is administered for a period of time of about 1 day to about 15 days.

69. The use of any one of claims 45 to 68, wherein the slowing/delaying cellular senescence comprises slowing/delaying cellular senescence of peripheral blood mononuclear cells.

70. The use of any one of claims 45 to 68, wherein the slowing/delaying cellular senescence comprises decreasing % of senescent peripheral blood mononuclear cells.

71. The use of any one of claims 45 to 68, wherein the slowing/delaying cellular senescence comprises decreasing at least about 25% of senescent peripheral blood mononuclear cells in a period of time of about 21 days.

72. The use of any one of claims 45 to 68, wherein the slowing/delaying cellular senescence comprises decreasing at least about 25% of senescent peripheral blood mononuclear cells in a period of time of about 14 days.

73. The use of any one of claims 45 to 68, wherein the slowing/delaying cellular senescence comprises decreasing at least about 25% of senescent peripheral blood mononuclear cells in a period of time of about 7 days.

74. The use of any one of claims 45 to 68, wherein the slowing/delaying cellular senescence comprises decreasing at least about 33% of senescent peripheral blood mononuclear cells in a period of time of about 21 days.

75. The use of any one of claims 45 to 68, wherein the slowing/delaying cellular senescence comprises decreasing at least about 33% of senescent peripheral blood mononuclear cells in a period of time of about 14 days.

76. The use of any one of claims 45 to 68, wherein the slowing/delaying cellular senescence comprises decreasing at least about 33% of senescent peripheral blood mononuclear cells in a period of time of about 7 days.

77. A method of using at least one compound chosen from compound of formula (I), compound of formula (II), compound of formula (III) and compound of formula (IV), said method comprising administering an effective amount of said at least one compound for cellular senescence prevention.

78. A method for slowing/delaying cellular senescence of a subject in need thereof comprising administering to the subject an effective amount of at least one compound chosen from compound of formula (I), compound of formula (II), compound of formula (III) and compound of formula (IV).

79. The method of any one of claims 77 to 78, wherein said at least one compound is said compound of formula (I).

80. The method of any one of claims 77 to 78, wherein said at least one compound is said compound of formula (II).

81. The method of any one of claims 77 to 78, wherein said at least one compound is said compound of formula (III).

82. The method of any one of claims 77 to 78, wherein said at least one compound is said compound of formula (IV).

83. The method of any one of claims 77 to 78, wherein said at least one compound is said compound of formula (I), said compound of formula (III) and said compound of formula (IV).

84. The method of any one of claims 77 to 78, wherein said at least one compound is said compound of formula (I) and said compound of formula (IV).

85. The method of any one of claims 77 to 78, wherein said at least one compound is said compound of formula (I) and said compound of formula (III).

86. The method of any one of claims 77 to 78, wherein said at least one compound is said compound of formula (III) and said compound of formula (IV).

87. The method of any one of claims 77 to 78, wherein said at least one compound chosen from compound of formula (I), compound of formula (II), compound of formula (III) and compound of formula (IV) is administered in combination with at least one lipid.

88. The method of any one of claims 77 to 876, wherein said at least one lipid and said at least one compound are administered simultaneously.

89. The method of any one of claims 77 to 87, wherein said at least one lipid and said at least one compound are administered separately.

90. The method of any one of claims 77 to 87, wherein said at least one compound chosen from compound of formula (I), compound of formula (II), compound of formula (III) and compound of formula (IV) is administered in combination with at least one polyphenol.

91. The method of any one of claim 90, wherein said at least one polyphenol and said at least one compound are administered simultaneously.

92. The method of any one of claim 90, wherein said at least one polyphenol and said at least one compound are administered separately.

93. The method of claim 90, 91 or 92, wherein said at least one polyphenol is chosen from flavonoids anthocyanidins, flavan-3-ols, epigallocatechin gallate, fisetin, flavonols, quercetin, flavanones, flavones, isoflavones, tannic acid, quercitannic acid, gallotannic acid and mixtures thereof.

94. The method of claim 90, 91 or 92, wherein said at least one polyphenol is chosen from epigallocatechin gallate, fisetin, quercetin, and mixtures thereof.

95. The method of any one of claims 90 to 94, wherein said compound chosen from compound of formula (I), compound of formula (II), compound of formula (III) and compound of formula (IV) is administered to a patient in need thereof at a suitable amount.

96. The method of any one of claims 90 to 94, wherein said compound chosen from compound of formula (I), compound of formula (II), compound of formula (III) and compound of formula (IV) is administered to a patient in need thereof in an amount of about 50 mg/day to about 5000 mg/day.

97. The method of any one of claims 90 to 94, wherein said compound chosen from compound of formula (I), compound of formula (II), compound of formula (III) and compound of formula (IV) is administered to a patient in need thereof in an amount of about 50 mg/day to about 500 mg/day.

98. The method of any one of claims 90 to 94, wherein said compound chosen from compound of formula (I), compound of formula (II), compound of formula (III) and compound of formula (IV) is administered to a patient in need thereof in an amount of about 1 mg/kg/day to about 100 mg/kg/day.

99. The method of any one of claims 90 to 94, wherein said compound chosen from compound of formula (I), compound of formula (II), compound of formula (III) and compound of formula (IV) is administered to a patient in need thereof in an amount of about 1 mg/kg/day to about 50 mg/kg/day.

100. The method of any one of claims 90 to 94, wherein said compound chosen from compound of formula (I), compound of formula (II), compound of formula (III) and compound of formula (IV) is administered to a patient in need thereof in an amount of about 2 mg/kg/day to about 20 mg/kg/day.

101. The method of any one of claims 90 to 101, wherein said compound chosen from compound of formula (I), compound of formula (II), compound of formula (III) and compound of formula (IV) is administered for a period of time of at least 1 month.

102. The method of any one of claims 90 to 101, wherein said compound chosen from compound of formula (I), compound of formula (II), compound of formula (III) and compound of formula (IV) is administered for a period of time of at least 4 months.

103. The method of any one of claims 90 to 101, wherein said compound chosen from compound of formula (I), compound of formula (II), compound of formula (III) and compound of formula (IV) is administered for a period of time of about 1 month to about 12 months.

104. The method of any one of claims 90 to 101, wherein said at least one polyphenol is administered to a patient in need thereof at a suitable amount.

105. The method of any one of claims 90 to 104, wherein said at least one polyphenol is administered to a patient in need thereof in an amount of about 50 mg/day to about 5000 mg/day.

106. The method of any one of claims 90 to 104, wherein said at least one polyphenol is administered to a patient in need thereof in an amount of about 50 mg/day to about 500 mg/day.

107. The method of any one of claims 90 to 104, wherein said at least one polyphenol is administered to a patient in need thereof in an amount of about 1 mg/kg/day to about 100 mg/kg/day.

108. The method of any one of claims 90 to 104, wherein said at least one polyphenol is administered to a patient in need thereof in an amount of about 1 mg/kg/day to about 50 mg/kg/day.

109. The method of any one of claims 90 to 104, wherein said at least one polyphenol is administered to a patient in need thereof in an amount of about 2 mg/kg/day to about 20 mg/kg/day.

110. The method of any one of claims 90 to 109, wherein said at least one polyphenol is administered for a period of time of at least 2 days.

111. The method of any one of claims 90 to 109, wherein said at least one polyphenol is administered for a period of time of at least 10 days.

112. The method of any one of claims 90 to 109, wherein said at least one polyphenol is administered for a period of time of about 2 days to about 15 days.

113. The method of any one of claims 90 to 109, wherein said compound chosen from compound of formula (I), compound of formula (II), compound of formula (III) and compound of formula (IV) is administered to a patient in need thereof for a period of time of about 1 to 12 months, then said administration is discontinued and subsequent to discontinuation, said at least one polyphenol is administered for a period of time of about 1 day to about 15 days.

114. The method of any one of claims 90 to 113, wherein the slowing/delaying cellular senescence comprises slowing/delaying cellular senescence of peripheral blood mononuclear cells.

115. The method of any one of claims 90 to 113, wherein the slowing/delaying cellular senescence comprises decreasing % of senescent peripheral blood mononuclear cells.

116. The method of any one of claims 90 to 113, wherein the slowing/delaying cellular senescence comprises decreasing at least about 25% of senescent peripheral blood mononuclear cells in a period of time of about 21 days.

117. The method of any one of claims 90 to 113, wherein the slowing/delaying cellular senescence comprises decreasing at least about 25% of senescent peripheral blood mononuclear cells in a period of time of about 14 days.

118. The method of any one of claims 90 to 113, wherein the slowing/delaying cellular senescence comprises decreasing at least about 25% of senescent peripheral blood mononuclear cells in a period of time of about 7 days.

119. The method of any one of claims 90 to 113, wherein the slowing/delaying cellular senescence comprises decreasing at least about 33% of senescent peripheral blood mononuclear cells in a period of time of about 21 days.

120. The method of any one of claims 90 to 113, wherein the slowing/delaying cellular senescence comprises decreasing at least about 33% of senescent peripheral blood mononuclear cells in a period of time of about 14 days.

121. The method of any one of claims 90 to 113, wherein the slowing/delaying cellular senescence comprises decreasing at least about 33% of senescent peripheral blood mononuclear cells in a period of time of about 7 days.

122. A composition comprising:
(i) at least one compound chosen from compound of formula (I), compound of formula (II), compound of formula (III) and compound of formula (IV); and
(ii) at least one lipid.

123. The composition of claim 122, wherein said at least one lipid is chosen from marine oil, fish oil, fish oil concentrate, re-esterified fish oil, re-esterified concentrated fish oil, fatty acids ethyl esters, fatty acids esters, monoglycerides, diglycerides, triglycerides and phospholipids.

124. The composition of any one of claims 122 to 123, wherein said at least one compound is said compound of formula (I).

125. The composition of any one of claims 122 to 123, wherein said at least one compound is said compound of formula (II).

126. The composition of any one of claims 122 to 123, wherein said at least one compound is said compound of formula (III).

127. The composition of any one of claims 122 to 123, wherein said at least one compound is said compound of formula (IV).

128. The composition of any one of claims 122 to 123, wherein said at least one compound is said compound of formula (I), said compound of formula (III) and said compound of formula (IV).

129. The composition of any one of claims 122 to 123, wherein said at least one compound is said compound of formula (I) and said compound of formula (IV).

130. The composition of any one of claims 122 to 123, wherein said at least one compound is said compound of formula (I) and said compound of formula (III).

131. The composition of any one of claims 122 to 123, wherein said at least one compound is said compound of formula (III) and said compound of formula (IV).

132. A composition comprising:
(i) at least one compound chosen from compound of formula (I), compound of formula (II), compound of formula (III) and compound of formula (IV); and
(ii) at least one polyphenol.

133. The composition of claim 132, wherein said at least one polyphenol is chosen from flavonoids anthocyanidins, flavan-3-ols, epigallocatechin gallate, fisetin, flavonols, quercetin, flavanones, flavones, isoflavones, tannic acid, quercitannic acid, gallotannic acid and the Blend of Fisetin/Quercetin (1:5), microalgae polyphenol, macroalgae polyphenol, plants polyphenol, protists polyphenol and pharmaceutically acceptable salts thereof.

134. The composition of claim 132, wherein said at least one polyphenol is chosen from flavonoids anthocyanidins, flavan-3-ols, epigallocatechin gallate, fisetin, flavonols, quercetin, flavanones, flavones, isoflavones, tannic acid, quercitannic acid, gallotannic acid and mixtures thereof.

135. The composition of claim 132, wherein said at least one polyphenol is chosen from epigallocatechin gallate, fisetin, quercetin, and mixtures thereof.

136. The composition of claim 132, wherein said at least one polyphenol is chosen from microalgae polyphenols, macroalgae polyphenols, plants polyphenols and protists polyphenols.

137. The composition of any one of claims 132 to 136, wherein said at least one compound is said compound of formula (I).

138. The composition of any one of claims 132 to 136, wherein said at least one compound is said compound of formula (II).

139. The composition of any one of claims 132 to 136, wherein said at least one compound is said compound of formula (III).

140. The composition of any one of claims 132 to 136, wherein said at least one compound is said compound of formula (IV).

141. The composition of any one of claims 132 to 136, wherein said at least one compound is said compound of formula (I), said compound of formula (III) and said compound of formula (IV).

142. The composition of any one of claims 132 to 136, wherein said at least one compound is said compound of formula (I) and said compound of formula (IV).

143. The composition of any one of claims 132 to 136, wherein said at least one compound is said compound of formula (I) and said compound of formula (III).

144. The composition of any one of claims 132 to 136, wherein said at least one compound is said compound of formula (III) and said compound of formula (IV).

145. The at least one compound of claim 16, 17 or 18, wherein said at least one polyphenol is chosen from microalgae polyphenols, macroalgae polyphenols, plants polyphenols and protists polyphenols.

146. The use of claim 45, 46 or 47, wherein said at least one polyphenol is chosen from microalgae polyphenols, macroalgae polyphenols, plants polyphenols and protists polyphenols.

147. The method of claim 90, 91 or 92, wherein said at least one polyphenol is chosen from microalgae polyphenols, macroalgae polyphenols, plants polyphenols and protists polyphenols.
